(19) United States
(12) Patent Application Publication
(10) Pub. No.: US 2016/0174898 A1
(43) Pub. Date: Jun. 23, 2016

(54) PHYSIOLOGICAL PARAMETERS MONITORING SYSTEM

(71) Applicants: EMEM UFOT UDOH, Maple Grove, MN (US); Tonya Sothan Heng, Maple Grove, MN (US); Trung Vu, Brooklyn Center, MN (US)

(72) Inventors: EMEM UFOT UDOH, Maple Grove, MN (US); Tonya Sothan Heng, Maple Grove, MN (US); Trung Vu, Brooklyn Center, MN (US)

(73) Assignee: ETON LABS, INC., Maple Grove, MN (US)

(21) Appl. No.: 14/573,236

(22) Filed: Dec. 17, 2014

(51) Int. Cl. A61B 5/00; A61B 5/0205; A61B 7/04; A61B 5/01; A61B 8/02

(52) U.S. Cl. CPC A61B 5/6807; A61B 5/0059; A61B 5/01; A61B 8/02; A61B 7/04; A61B 5/7405; A61B 5/0022; A61B 5/747; A61B 5/02055; A61B 2560/0214; A61B 2560/0242

(57) ABSTRACT

A system and method for monitoring the physiological conditions of a subject using a battery powered physiological monitoring device configured to communicate through wireless communication with other electronic devices such as mobile devices, computers and networks is presented. The invention includes a plurality of physiological sensors integrated or built into a sock, wristband or as a standalone unit with the ability to process and stores the physiological data collected and provide useful information to the subject, monitoring equipment or health care professional.

### BACKGROUND

The present inventions relates to physiological parameters monitoring, more specifically to systems and methods for monitoring the physiological conditions of a subject.

In a society that is getting increasingly conscious of health parameters and presented with more options to understand and study when, where and how to do more or less with the resources available regarding the health of the ones in our care, the use of thermometers, scopes and other equipment for such data is increasing. Society also reacts negatively when that collection of data is left with some measure of discomfort or is unusually intrusive. The health parameters of infants are especially important as they do not possess the capacity to voice out their ailments or bothers as do adults. Adults also prefer use of monitors that provide information that can be kept in the privacy of their reach without seeking the aid of third parties at every whim.

When there is need to find out how healthy someone is, preferably via vital signs, most people have to go to clinics, hospitals or similar facilities for tests and measurements. Sometimes, getting to such facilities depend on time of day or night, resources at the disposal of the intended person or even time to be allocated to such process. If the intended person is a baby, the rigor of taking the baby to a facility and time of such need create some inconvenience for the parties involved. The more information needed at each instance, the more complicated or engaged the inconvenience would typically be. As an example, if more parameters are needed to be monitored or checked, the more complicated the apparatus or setup it typically is for such information to be collected.

With the increasing need for monitoring human health and the everyday physiological activity, a robust, reliable, unobtrusive and comfortable way to acquire these physiological parameters is needed. The capacity to transfer or store such information is also an expectation. Such an apparatus and system are provided in the present invention and claimed herein.

### SUMMARY

In one embodiment the present invention provides a physiological parameters monitoring apparatus having a wearable unit that includes at least a sensor, audio means, data transmitting means and a data receiving means wherein the unit can suitable communicate with a database network to display or transmit the parameters of a wearer.

In another embodiment of the present invention, the physiological monitoring device of the present invention independently assesses the physiological conditions of the user and provides appropriate warning messages in instances of abnormal readings.

It is also an object of the present invention that the monitoring device of the present invention can be placed next to human skin without an adverse risk to the skin of the wearer. Such adverse outcomes include irritation of the skin, burning or otherwise marking the skin of the wearer.

An adult female using the physiological monitoring device of the present invention may be able to determine through their basal temperatures, an optimum ovulation period or when she is ovulating.

When used on the stomach of a pregnant woman, the present invention provides opportunities to monitor the physiological conditions of a fetus, pregnant woman or gravida for use in determining pre-natal conditions for optimum healthcare management.

According to the present invention, data collected through the application of the physiological monitoring device may preferably be transmitted to a base, received by that base for use in a database or network. The network may then be made available to end users that may include servers, caregivers, emergency services, physicians, educators or other third parties.

It is reasonable to expect use of conductive materials on the skin for optimizing the transmission of data and information without an excess light or heat generated.

### BRIEF DESCRIPTION OF THE DRAWINGS

FIG. 1A is a side perspective view of an embodiment of the physiological monitoring system showing the foot wrap according to the present invention.

FIG. 1B is a top perspective view of an embodiment of the physiological monitoring system according to the present invention.

FIG. 1C is a rear perspective view of an embodiment of the physiological monitoring system according to the present invention, showing the foot wrap about the heel of the user.

FIG. 2 is a schematic view of an embodiment of the physiological monitoring system according to the present invention showing the monitor and communication system.

FIG. 3 is another schematic view of an embodiment of the physiological monitoring system according to the present invention showing the monitor and communication system.

FIG. 4 is another schematic view of an embodiment of the physiological monitoring system according to the present invention showing aspects of the monitoring unit.

FIG. 5 is a flow chart of an embodiment of the physiological monitoring system according to the present invention.

FIG. 6 is another schematic view of an embodiment of the physiological monitoring system according to the present invention showing the monitor wrapped around the stomach of an expectant mother, wrist or arm of a user and communication system.

FIG. 7 is another schematic view of an embodiment of the physiological monitoring system according to the present invention showing the monitor wrapped around the stomach of an expectant mother, wrist or arm of a user and communication system.

FIG. 8A is a view of an embodiment of a monitor usable in the present invention showing a removable port and an extended adapter.

FIG. 8B is a side perspective view of an embodiment of a monitor usable in the present invention.

FIG. 8C is another perspective view of an embodiment of a monitor usable in the present invention showing a removable port and an extended adapter.

### DETAILED DESCRIPTION

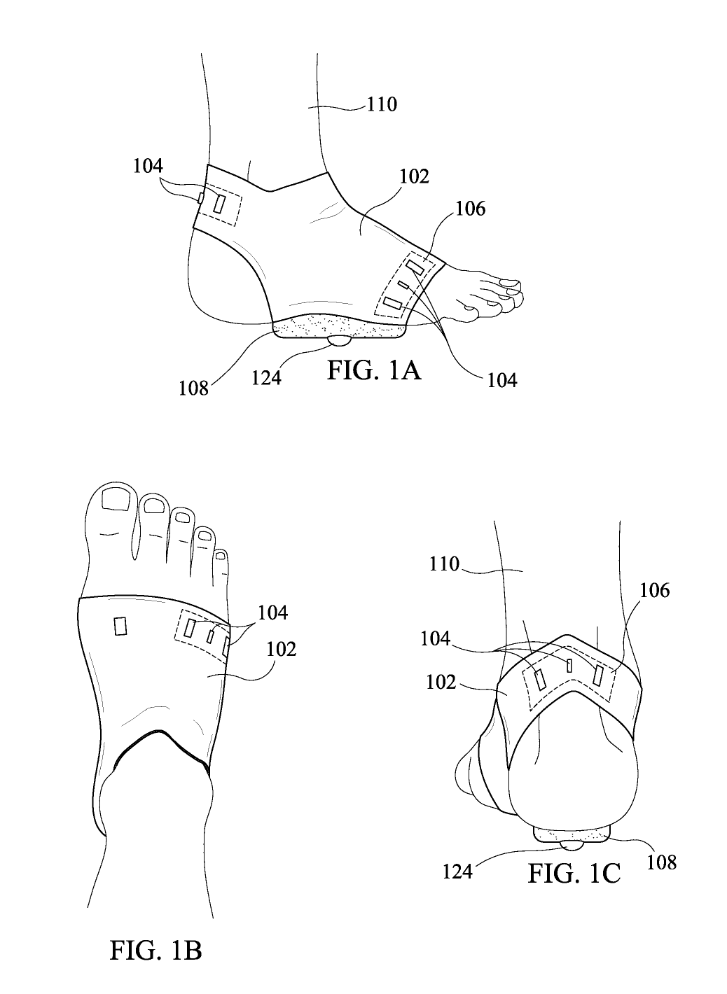

Referring to FIGS. 1A-C, a physiological monitoring unit according to the present invention is presented having a sock, cloth or wearable device body 102 comprising at least a sensor or measuring device 104, said sensor or monitoring device preferably or operably included in a patch 106, which monitoring device may include other sensing units and a physiological monitoring unit 108. The monitoring unit 108 is preferably suitable for use on an intended object or human and may further comprise sensing devices 124. In a preferred embodiment, the wearable device may preferably be formed to be worn as a sock, wrap, patch, cloth, ankle or foot gear, suitable to be used on a leg 110 or appendage of the object of interest being monitored. Given the multiple use of the physiological monitor of the present invention, it is recognizable that other appendages or body part may benefit from the use or application of this invention.

The sensors 104 may preferably include at least a light emitting unit and at least a light sensing unit, said light units embodied in a patch 106 which is preferably made from a skin protective material at these are applicable onto a skin of an individual. These materials may be formed from breathable cotton cloths or other suitable material as are good and functional to be used for the intended purpose. Depending on need and application, the sensors 104 of the present invention may preferably include temperature or other sensors to meet the need of the intended process. The monitoring unit of the present invention is preferably and operably adapted to fit the appendage or leg of the object being monitored and further suitable to be used to obtain pertinent data to assess the physiological parameters of the subject monitored. Such data need may include temperature, blood pressure, oxygen intake and level, heart rate, pulse and other needed information.

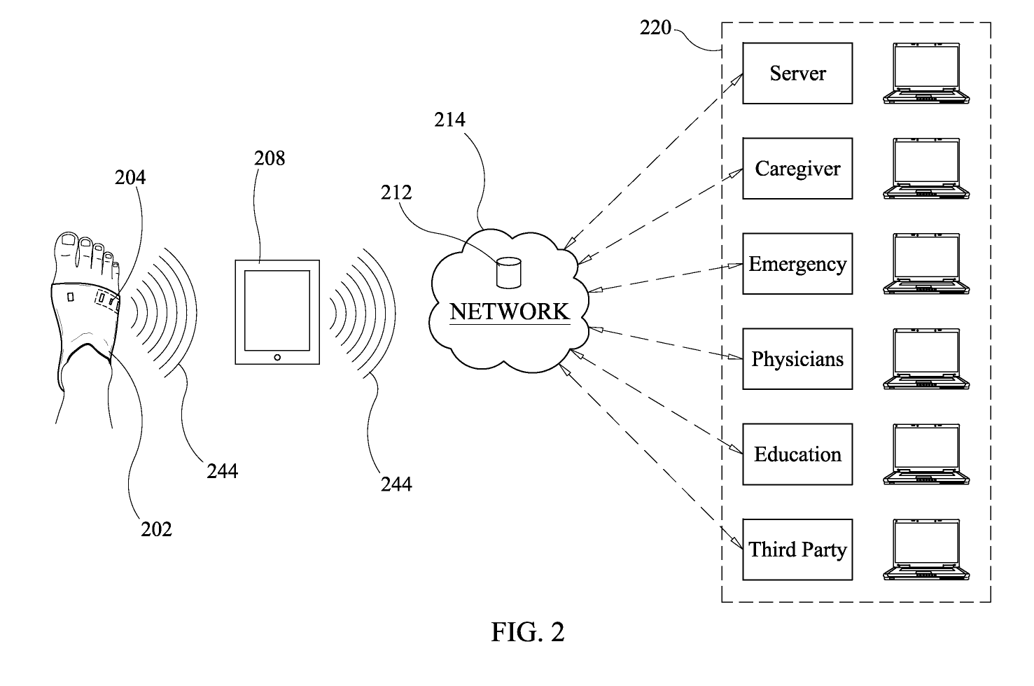

According to the present invention, FIG. 2 preferably illustrates an information collection system with sensors or sensory units 204, a transmitting means 244 for communicating with a base station 208, a further transmitting means 244 for communication with a network or cloud system 214, wherein said network or cloud system comprising a database 212 and a plethora of user points 220 for servers, caregivers, emergency services, physicians, educators or third party. The transmitting means may be wireless or wired (not shown), adaptable to be used to obtain the needed information for the monitoring of a subject according to the present invention. In some instances, wireless or wired transmission of the data using a base station 208 may include an enhancement to assure data connectivity with the network base or collecting station. It is reasonable to include a power supply unit, booster or charging mechanism to provide such enhancement for the assured data transfer between locations according to the present invention. As indicated, base station 208 preferably includes a mobile connectivity as is known in the art for use in sharing or transferring data to a network system 214 that may or may not include use of a database 212. Data processing or management may preferably take place within the network 214 or database 212 in concert with users and outlets such as servers, caregivers, emergency services, physicians, educators and other third parties, collectively 220. Those skilled in the art are typically conversant with such systems and the application of such services to the management and use of the data or information collected.

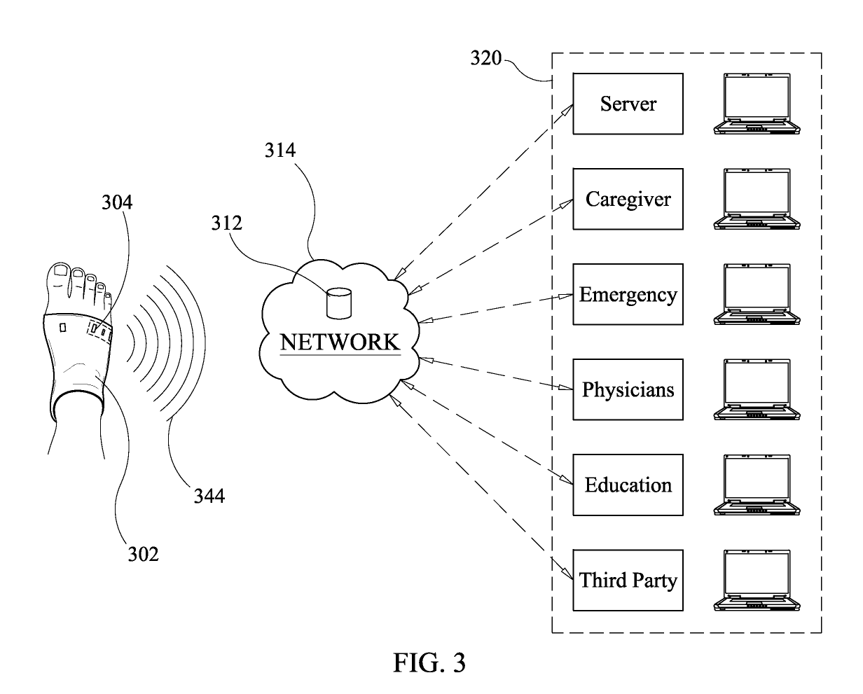

In yet another embodiment of the present invention and in the manner described above, FIG. 3 preferably illustrates an information collection system with sensors or sensory units 304, a transmitting means 344 for communication with a network or cloud system 314, wherein said network or cloud system comprising a database 312 and a plethora of user points 320 for servers, caregivers, emergency services, physicians, educators or third party. In this embodiment, the data or information collected is directly communicated or transferred to the network or database without an intervening step or process of communication with a base station. As disclosed above, the communication system of the instant invention may be wireless or wired depending on application and circumstance of use.

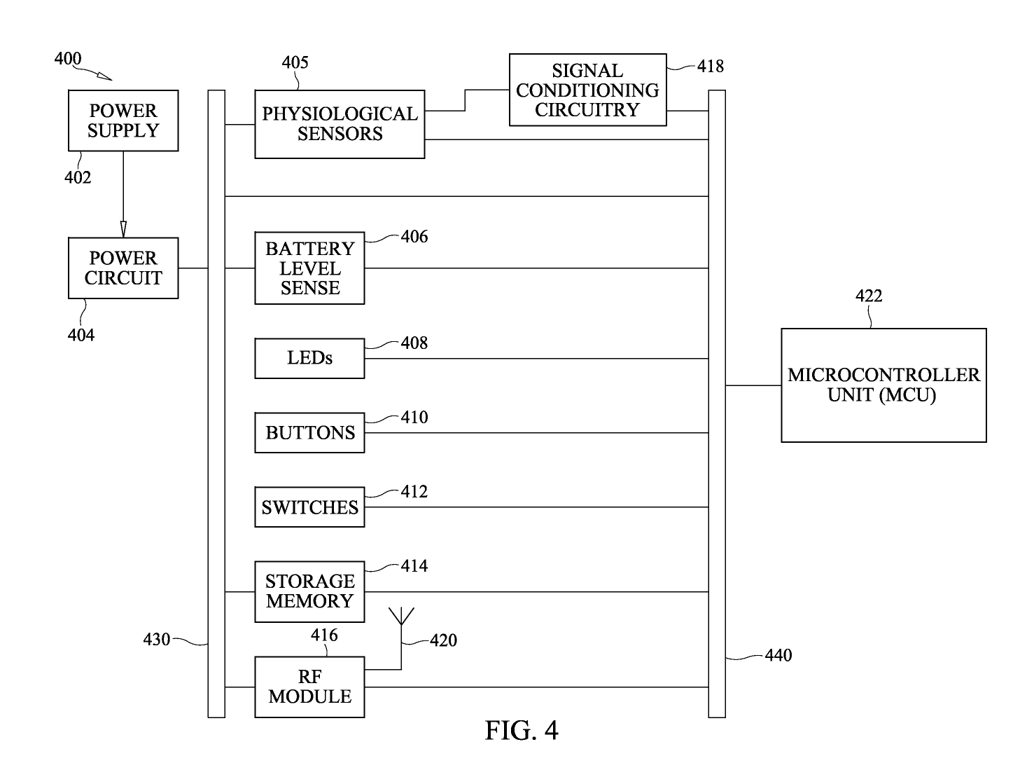

FIG. 4 illustrates a block schematic diagram of a preferred embodiment of the monitoring unit according to the present invention 400 illustratively showing a power supply 402 linked to a power circuit 404 including a power bus 430, a set of physiological sensors 405, a signal conditioning circuitry 418, a battery or power level sensor 406, light emitting diodes 408, user managed buttons or keys 410, user managed switches 412, a storage memory 414, a radio-frequency module 416, an antenna 420, a communication bus 440, and at least a microcontroller unit 422. The physiological sensors can and may be very broad to include most advances in medical science and technology to non-invasively collect pertinent data for the assessment of the user's condition. Such sensors include but are not limited to electro-cardiogram (ECG), motion, movement, orientation, accelerometer, heart rate, blood glucose, respiration rate, temperature, weight, blood pressure, activity level, switches, indicators and other buttons. A signal conditioning circuitry may be incorporated for use in filtering, amplifying and/or isolating the output from or input into the physiological sensors. The RF module 416 of the present invention may be wired or wireless for use as cellular, Wireless Fidelity (WiFi), Bluetooth, Ethernet, or other systems and protocol as known to those skilled in the art. The power supply 402 according to the present invention may preferably be wireless or battery powered.

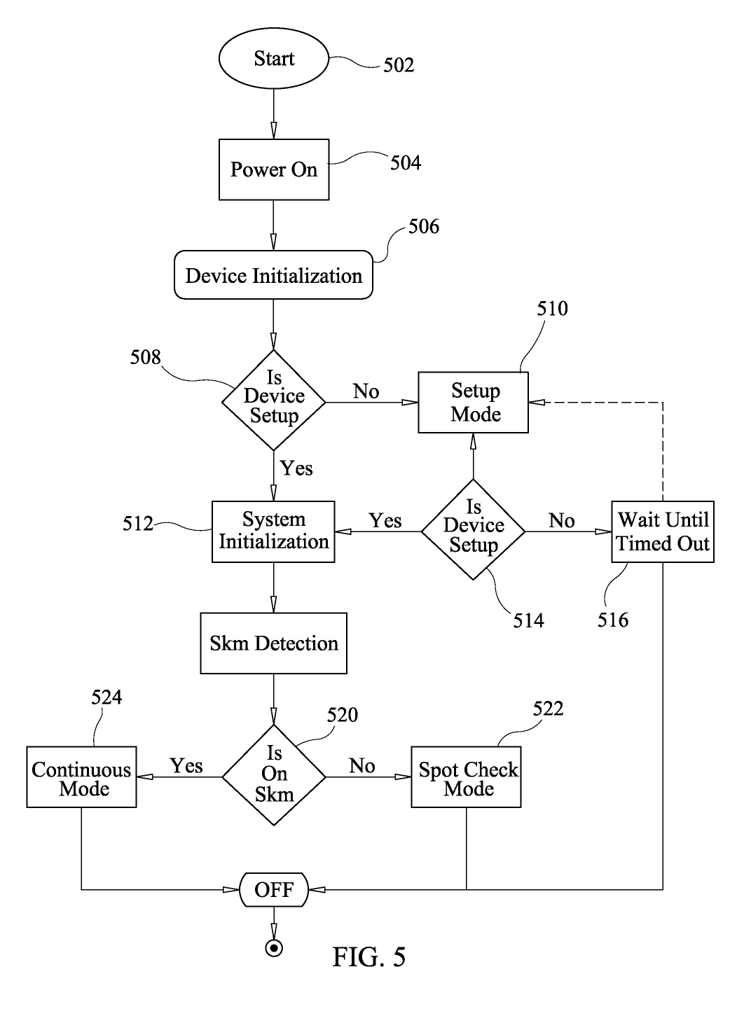

A flowchart of a process, according to an embodiment of the present invention is presented in FIG. 5 indicating a start 502 wherein the system is powered on 504 and the device initialized 506. Upon determination that the device is set up 508 for operation, a system initialization 512 is preferably performed pending a detection of placement of device. If the device detects placement on skin of user or person monitored, a check 520 to evaluate if monitoring is continuous 524 or spot check 522 is accomplished. At each stage in the process, if the system is not set up for operation 514, a check to determine status and a correction to reset same for operation is preferably conducted. The process sequence according to the present invention includes self-correcting and self-adapting steps to ensure and assure monitoring when needed.

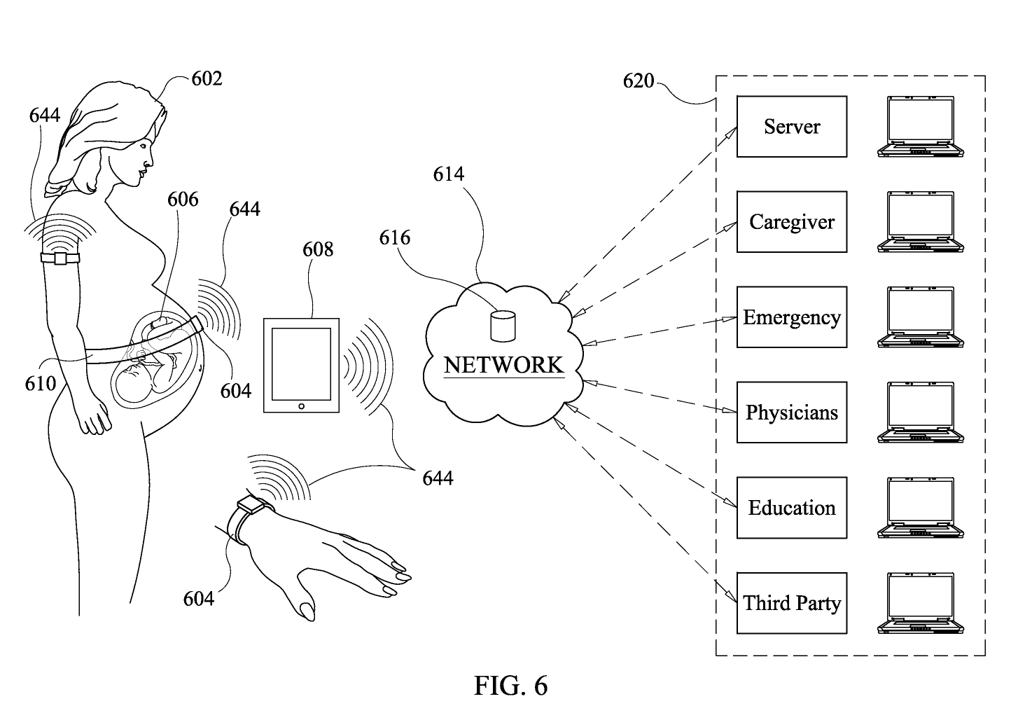

Now referring to FIG. 6, an expectant mother shown in 602, with a monitoring unit on the arm, wrist or belly 604, a fetus 606, a transmitting means 644, a base station 608, another transmitting unit 644, a network or cloud 614 with a database 616, in communication with users 620 that may include servers, caregivers, emergency service providers, physicians, educators or other third party (ies). As discussed above, the physiological sensing or monitoring unit 604 may be worn around any potential source of information to assess the physiological status of the fetus or object of interest. A list of prenatal or fetal monitoring configurations may be formed or established using the equipment and formations according to the present invention. The monitoring units 604 when used by a woman may be configured to provide physiological information suitable to indicate fertility opportunities such as peak ovulation times for the woman.

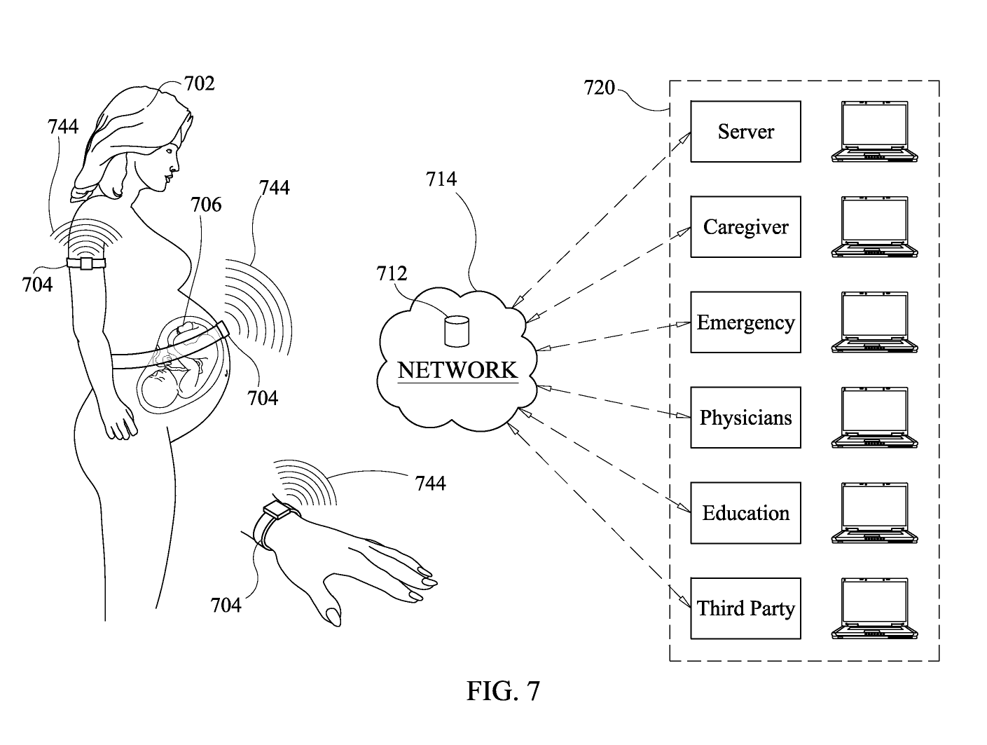

In yet another embodiment and in furtherance of the discussion above, shown in FIG. 7, an expectant mother shown in 702, with a monitoring unit on the arm, or wrist or belly 704, a fetus 706, a transmitting unit 744, a network or cloud 714 with a database 712, in communication with users 720 that may include servers, caregivers, emergency service providers, physicians, educators or other third party (ies). According to the present embodiment, data or information collected may preferably be directly communicated or transferred via a transmitting means 744 to a network 714 or database 712 without the need for an intervening step or process of communication with a base station 708. As discussed above, the physiological sensing or monitoring unit 704 may be worn around any potential source of information to assess the physiological status of the fetus or object of interest. A list of prenatal or fetal monitoring configurations may be formed or established using the equipment and formations according to the present invention. The monitoring units 704 when used by a woman may be configured to provide physiological information suitable to indicate fertility opportunities such as peak ovulation times for the woman.

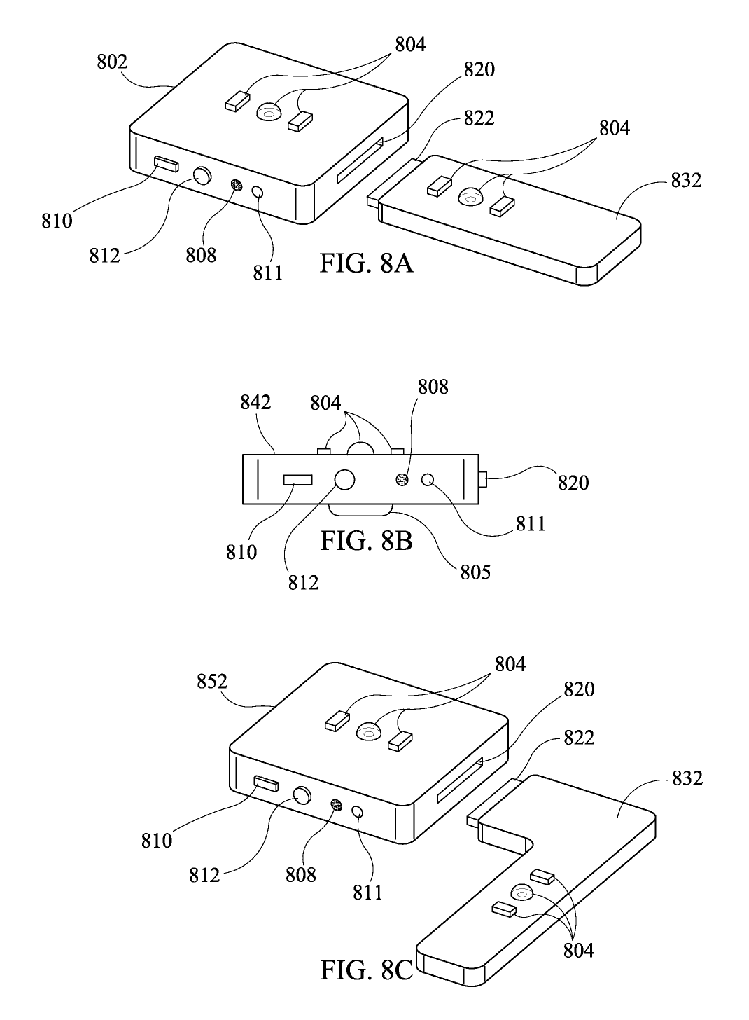

In FIGS. 8A-C perspectives of a monitoring unit 802 showing light emitting and sensing sensors or diodes 804, 842, 852, audio means 812, acoustic ultrasonic sensor 805, buttons 810, temperature sensors, battery level sensors and more for use in monitoring the physiological parameters of interest according to the present invention. Also included is a detachable connector 832 having some additional or duplicated sensors 804 and connector 822. Said connector 822 may be usable as a charging mechanism for the monitoring unit and is preferably adaptable to connect with the monitoring unit at 820. Said audio means 812 having hardware circuitry to detect, play and record sound signals.

In use, the physiological parameters monitoring unit of the present invention provides equipment useful to monitor needed parameters of a subject. The equipment is preferably adaptable to conform to the shape of a foot or appendage of a subject—for infants may be usable as a sock or a wrap for an adult. The equipment is useful to monitor the neonate physiological conditions and can equally be adapted to provide vital signs data for others including adults. In other applications, the physiological monitoring unit may be used to monitor the prenatal conditions of fetuses or expectant mothers, such conditions including heart rates, activity level and other desired conditions. The monitoring unit of the present invention may be usable to monitor the basal temperature of a woman to determine the ovulation conditions and other conditions of interest.

The monitoring unit of the present invention may be adaptable for use as a standalone equipment wherein data collected via wireless or radio frequency means can be transmitted to a database or secondary monitoring station or professional which may include a server, caregiver, emergency units, physicians and others. It is reasonable to include research and other users such as parents as users of information or data available from the equipment of the present invention.

Several parameters may be monitored with the equipment of the present invention, including without limitation, electrocardiograph, respiratory rate, pulse rate, blood pressure, body temperature, posture, skin conductance and arterial hemoglobin, oxygen saturation, mixed venous oxygenation, among others. Adaptation for monitoring these parameters may include flex and conformable material such as socks, pliable sensors that are detachable and connectable to the monitoring units. Other variations as may be known or later formed by those skilled in the art are contemplated under this invention.

The preferable material usable for the monitoring unit of the present invention should be soft, pliable and of such quality to not irritate or otherwise bother the skin of the user. Typical materials include silicone that may optimally not irritate the human skin. Other materials of similar quality or performance may be used. For infants and others with tender skin, material such as cotton or socks that can be made to conform to the shape of the application location (feet or hand) with the focus that little or no irritation occurs.

Conductive material is preferable for use in the present invention. Materials such as medically acceptable conductive material that would not store heat or cause heat to be generated at the point of application are preferred.

The transmission of data from the physiological monitoring unit can be done wirelessly to a base station, network or internet. Computers and other electronic equipment that may be useful in the data transmission may be adapted to efficiently transfer data to other users or stations without disruption or distortion.

During operation, the physiological sensors of the present invention may include heat flux, speech features, electrocardiograph, respiratory rate, pulse rate, blood pressure, skin temperature, posture, electro-dermal response and arterial hemoglobin, oxygen saturation, mixed venous oxygenation, among others. The signals from these sensors may then be filtered and converted into corresponding electric signals obtainable by a microcontroller unit for processing into corresponding digital signals via a communication bus. Such and other variations of this data and signal transformation are anticipated according to the scope of the present invention.

When optical sensors comprising light emitting and sensing diodes are incorporated according to the present invention, a peak emission wavelength of no more than 660 nanometers is anticipated. An infrared emitter is also anticipated at no more than 940 nanometers. An optimal distance is preferably selected for the light emitting and light sensing diodes for reliable alternating current signal. Such distance may be less than 10 millimeters (mm). Depending on need and application, a multi-wavelength light emitting diodes ranging from about 660 to 940 nanometers may be incorporated to identify other blood physiological component. It is reasonable to optimize the circuitry to amplify the light sensing diode output and delineate between alternating current and direct current components. At each stage in the operation, those skilled in the art are able to optimize the filtration of data and amplification of same, where needed and applicable.

Other applications of the present invention include processes to reset and power off the units by the wearer or user. Data storage for the sensed physiological information may be stored on memory sticks or flash card or other non-volatile memory equipment. The Radio Frequency (RF) module usable according to the present invention provides communication between the physiological monitoring unit and the wireless or wired network, providing information that is transferable via antenna, Bluetooth, Wireless-Fidelity (Wi-Fi), Zigbee,Voice-Over Internet Protocol (VoIP), Cellular network, Mobile-to-Mobile (M2M), or other now or later known processes.

The present invention presents a novel means and is suitable to use wireless technology to transmit physiological data. Also, according to the present invention, attributes such as sleep, temperature, movements, positions, sounds, ultrasonic fetal heart rate monitor (FHRM) can be monitored as needed. The output of the present invention can be controlled or managed from an external network wherein other associated users can receive the data and updates as appropriate, including setting up alarms for optimal care.

The use of the demonstrated embodiments is not deemed a limitation of the scope of this invention. Other designs are practical and plausible, including design changes to accommodate new processes or conditions to increase efficiency and effectiveness of operation.

Having thus described the preferred embodiments of the present invention, those of skill in the art will readily appreciate that the teachings found herein may be applied to yet other embodiments within the scope of the claims hereto attached. The complete disclosure of all patents, patent documents and publications are incorporated herein by reference as if individually incorporated.

What is claimed:

1. A monitoring apparatus, said apparatus comprising:
a monitoring unit having at least a sensor
audio means
transmitting means
receiving means
wherein the monitoring unit is suitable to communicate physiological parameters with a computer network to display or transmit said physiological parameters of a subject.

2. The monitoring apparatus of claim 1, wherein the monitoring unit is coupled to an article of clothing that fits like a sock on the appendage of a subject.

3. The monitoring apparatus of claim 1, wherein the monitoring unit is coupled to an article of clothing that fits like a body wrap around the foot or wrist or arm of a subject.

4. The monitoring apparatus of claim 1, wherein the monitoring unit is adaptable for skin contact to monitor the physiological parameter of a subject.

5. The monitoring apparatus of claim 1, wherein the monitoring unit is adaptable for skin contact as a belt around the stomach of a subject.

6. The monitoring apparatus of claim 1, wherein the monitoring unit is adaptable for skin contact as an arm or foot wrap or wristband.

7. The monitoring apparatus of claim 1, wherein the monitoring unit includes a battery or a self-charging means.

8. The monitoring apparatus of claim 1, wherein the sensor includes at least a light emitting diode.

9. The monitoring apparatus of claim 1, wherein the sensor includes at least a light sensing diode.

10. The monitoring apparatus of claim 1, wherein the sensor includes at least a sensor adapted to measure temperature.

11. The monitoring apparatus of claim 1, wherein the sensor includes at least an acoustic or ultrasonic or piezocrystal sensor for determining the heart rate of a subject.

12. The monitoring apparatus of claim 1, wherein the sensor includes at least an audio sensor for determining sound or noise signature of a subject environment.

13. The monitoring apparatus of claim 1, wherein the audio means is incorporated in the monitoring unit.

14. The monitoring apparatus of claim 1, wherein the audio means is operably to play sound signal to a subject.

15. The monitoring apparatus of claim 1, wherein the audio means is operably to record sound.

16. The monitoring apparatus of claim 1, wherein the transmitting means is incorporated in the monitoring unit.

17. The monitoring apparatus of claim 1, wherein the transmitting means is operably linked to an external base station.

18. The monitoring apparatus of claim 1, wherein the transmitting means is in communication with a network.

19. The monitoring apparatus of claim 1, wherein the receiving means is adaptable to receive data from the sensor.

20. The monitoring apparatus of claim 1, wherein the receiving means is adaptable to receive data from a network.

21. The monitoring apparatus of claim 1, wherein the monitoring unit is in communication with the base station, the network and end users.

22. The monitoring apparatus of claim 21, wherein the users are selected from servers, caregivers, emergency services, physicians, educators and similar users.

23. A method of monitoring the physiological parameters of a subject comprising:
using a monitoring apparatus having at least a processor, a sensor, an audio means, a transmitting means, a receiving means, wherein the monitoring apparatus is suitable to receive data from the skin of a user, receive data from a network and transmitting same to a network for users.
